(19) United States
(12) Patent Application Publication
(10) Pub. No.: US 2015/0133827 A1
(43) Pub. Date: May 14, 2015

(54) METHOD, APPARATUS, AND ULTRASOUND SYSTEM FOR INCREASING TEMPERATURE OF REGION OF INTEREST BY USING WEARABLE ULTRASOUND IRRADIATION APPARATUS

(71) Applicants: SAMSUNG ELECTRONICS CO., LTD., Suwon-si (KR); SAMSUNG LIFE PUBLIC WELFARE FOUNDATION, Seoul (KR)

(72) Inventors: Joonho SEO, Suwon-si (KR); Hotaik LEE, Suwon-si (KR); Wonchul BANG, Suwon-si (KR); Jungbae KIM, Suwon-si (KR); Youngsun KIM, Seoul (KR)

(73) Assignees: SAMSUNG LIFE PUBLIC WELFARE FOUNDATION, Seoul (KR); SAMSUNG ELECTRONICS CO., LTD., Suwon-si (KR)

(21) Appl. No.: 14/535,821

(22) Filed: Nov. 7, 2014

(30) Foreign Application Priority Data
Nov. 8, 2013 (KR) 10-2013-0135845

(51) Int. Cl. A61N 7/02; A61B 8/08; A61B 8/00; A61B 8/14

(52) U.S. Cl. CPC A61N 7/02; A61B 8/14; A61B 8/5223; A61B 8/4227
USPC 601/3

(57) ABSTRACT

An ultrasound irradiation apparatus includes first transducers configured to irradiate first ultrasound energy upon an object with respect to a potential diagnosis and to receive an echo signal which corresponds to the irradiated first ultrasound energy, second transducers configured to irradiate second ultrasound energy upon a region of interest which is determined based on a result of processing of the received echo signal, and a contact pad configured to facilitate a contact between the first transducers and the object, and to facilitate a contact between the second transducers and the object.

### CROSS-REFERENCE TO RELATED APPLICATIONS

This application claims priority from Korean Patent Application No. 10-2013-0135845, filed on Nov. 8, 2013, in the Korean Intellectual Property Office, the disclosure of which is incorporated herein by reference in its entirety.

### BACKGROUND

1. Field

Exemplary embodiments relate to a method, apparatus, and ultrasound system for increasing a temperature of a region of interest by using a wearable ultrasound irradiation apparatus.

2. Description of the Related Art

Ultrasound may be irradiated on an object and an ultrasound image of a region of interest to be set in the object may be generated by using an echo signal reflected from the object. At this time, the ultrasound image of the region of interest may include a temperature image that represents a temperature of a cross-section of the region of interest or a brightness (B)-mode image that represents brightness of the cross-section of the region of interest. In addition, a travel speed of an ultrasound signal for generating the ultrasound image varies with a temperature of a medium.

Conversely, a method for irradiating high-intensity focused ultrasound (hereinafter, referred to as HIFU) on a tumor (focus) to be cured to noninvasively cure the tumor is studied. At this time, the HIFU irradiated on the tumor may focally destroy or necrotize the tumor and may increase a temperature of the tumor so that medicine may smoothly penetrate into the tumor.

### SUMMARY

Provided are a method, apparatus, and ultrasound system for increasing a temperature of a region of interest by using a wearable ultrasound irradiation apparatus.

Provided is a computer readable recording medium in which programs to be executed by a computer are recorded.

Additional aspects will be set forth in part in the description which follows and, in part, will be apparent from the description, or may be learned by practice of the presented exemplary embodiments.

According to an aspect, an ultrasound irradiation apparatus includes first transducers configured to irradiate first ultrasound energy upon an object and to receive an echo signal which corresponds to the irradiated first ultrasound energy, second transducers configured to irradiate second ultrasound energy upon a region of interest which is determined based on a result of processing of the received echo signal, and a contact pad configured to facilitate a contact between the first transducers and the object, and to facilitate a contact between the second transducers and the object.

According to another aspect, a method for irradiating ultrasound energy upon a region of interest includes irradiating first ultrasound energy upon an object and receiving an echo signal which corresponds to the irradiated first ultrasound energy, determining a region of interest on which the second ultrasound energy is to be irradiated by using the received echo signal, determining a threshold temperature of the region of interest to be reached as a result of the second ultrasound energy being irradiated, and generating a sufficient amount of the second ultrasound energy to be irradiated upon the region of interest by using information relating to a position of the region of interest and information relating to the determined threshold temperature.

According to another aspect, a computer readable storage medium includes a non-transitory computer readable storage medium storing a computer program for executing the above-described method in a computer.

According to another aspect, an ultrasound system includes an ultrasound irradiation apparatus which includes first transducers configured to irradiate first ultrasound energy upon an object and to receive an echo signal which corresponds to the irradiated first ultrasound energy and second transducers configured to irradiate second ultrasound energy upon a region of interest and an ultrasound processing apparatus configured to determine the region of interest upon which the second ultrasound energy is to be irradiated by using the received echo signal, to determine a threshold temperature of the region of interest to be reached as a result of the second ultrasound being irradiated, to generate the second ultrasound energy by using information which relates to a position of the determined region of interest and information which relates to the determined threshold temperature, and to transmit a signal which corresponds to the generated second ultrasound energy to the ultrasound irradiation apparatus.

According to one or more exemplary embodiments, the ultrasound irradiation apparatus in which the flat transducers are arranged is adhered to the object in order to irradiate the second ultrasound energy so that a large steering range may be secured. Therefore, the second ultrasound energy may be irradiated upon the region of interest regardless of a size and volume of the region of interest, including a tumor.

In addition, the ultrasound irradiation apparatus is implemented in the form of a pad so that the second ultrasound energy may be accurately irradiated upon the region of interest even when the object moves. In addition, the ultrasound system with increased mobility is implemented so that convenience of the object (for example, a patient) may be increased.

In addition, the ultrasound irradiation apparatus has various sizes so that the patient may be cured regardless of the size and position of the region of interest. In addition, since the temperature of the region of interest may be increased to the predetermined threshold temperature as the second ultrasound energy is being irradiated, it is possible to facilitate an effective penetration of a medicine which is dependent on a temperature into the region of interest.

In addition, since the ultrasound irradiation apparatus is implemented by a material that is not displayed in a magnetic resonance (MR) image, it is possible to generate an image of the region of interest and to cure the region of interest together with the MRI apparatus.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a block diagram illustrating an example of an ultrasound system, according to an exemplary embodiment;

FIG. 2 is a view illustrating an example of an ultrasound irradiation apparatus, according to an exemplary embodiment;

FIG. 3 is a view illustrating an example of implementing an ultrasound irradiation apparatus, according to an exemplary embodiment;

FIG. 4 is a block diagram illustrating an example of an ultrasound processing apparatus, according to an exemplary embodiment;

FIG. 5 is a block diagram illustrating another example of an ultrasound processing apparatus, according to an exemplary embodiment;

FIG. 6 is a block diagram illustrating another example of an ultrasound system, according to an exemplary embodiment;

FIG. 7 is a flowchart illustrating an example of a method for irradiating ultrasound on a region of interest, according to an exemplary embodiment; and

FIG. 8 is a view illustrating second transducers, according to an exemplary embodiment.

### DETAILED DESCRIPTION

Reference will now be made in detail to exemplary embodiments, examples of which are illustrated in the accompanying drawings. In this regard, the present exemplary embodiments may have different forms and should not be construed as being limited to the descriptions set forth herein. Accordingly, the exemplary embodiments are merely described below, by referring to the figures, to explain aspects of the present disclosure.

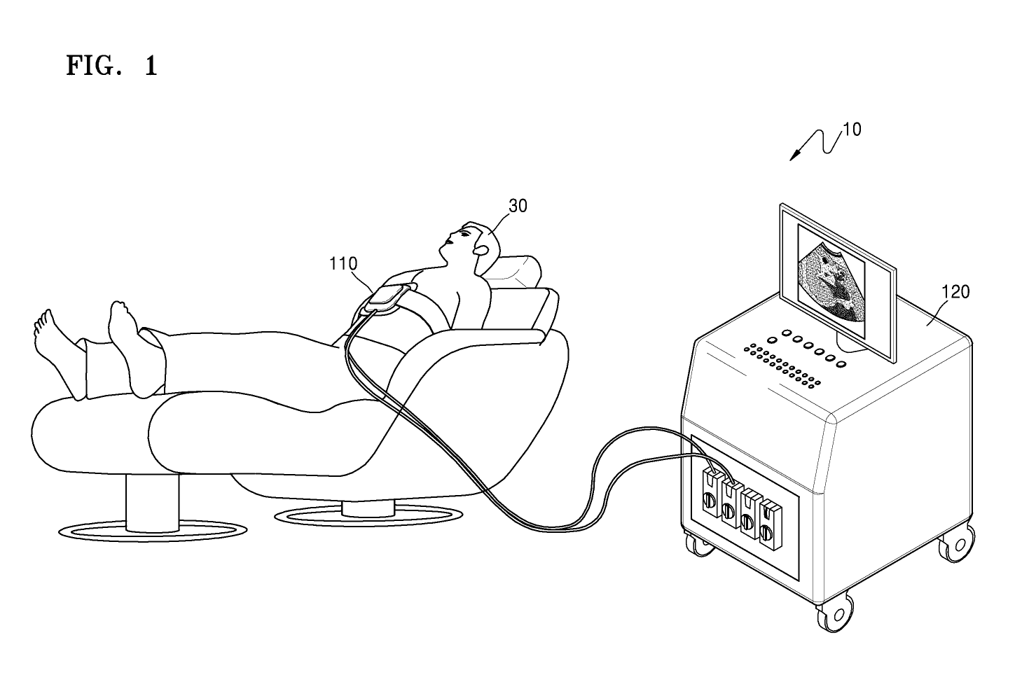

FIG. 1 is a block diagram illustrating an example of an ultrasound system, according to an exemplary embodiment.

Referring to FIG. 1, an ultrasound system 10 includes an ultrasound irradiation apparatus 110 and an ultrasound processing apparatus 120. Here, the ultrasound irradiation apparatus 110 may be adhered to a part of an object 30.

The ultrasound irradiation apparatus 110 irradiates first ultrasound energy (also referred to herein as “diagnostic ultrasound energy”) upon the object 30 (for example, a patient) and receives an echo signal which corresponds to the diagnostic ultrasound energy. In particular, the ultrasound irradiation apparatus 110 irradiates the diagnostic ultrasound energy upon a part of the object 30 to which the ultrasound irradiation apparatus 110 is adhered. Then, the ultrasound irradiation apparatus 110 receives the echo signal obtained as a result of the diagnostic ultrasound energy being reflected from the inside of the object 30. Here, the diagnostic ultrasound energy may include ultrasound energy to be used for generating an image that represents shapes of internal tissues of the object 30 and may include ultrasound energy to be used for generating an image that represents a change in temperature of a region of interest.

The ultrasound irradiation apparatus 110 irradiates second ultrasound energy (also referred to herein as “therapeutic ultrasound energy”) upon the region of interest which is determined in the object 30. Here, the region of interest may include a lesion of the object 30, but is not limited to the lesion of the object 30. In addition, the therapeutic ultrasound energy may include ultrasound energy to be used for increasing a temperature of the region of interest to a predetermined threshold temperature in order to facilitate a penetration of medicine into the region of interest. For example, the therapeutic ultrasound energy may include high-intensity focused ultrasound (HIFU). However, the therapeutic ultrasound energy is not limited to the HIFU, and may include any focused ultrasound which is irradiated in a similar manner as the HIFU.

When the ultrasound irradiation apparatus 110 continuously irradiates the therapeutic ultrasound energy upon a specific position in the region of interest, temperatures of a tissue positioned in the specific position and a peripheral tissue are increased. When it is assumed that medicine that penetrates into the inside of the object 30 at a specific temperature (for example, A° C.) is administered and that the temperature of the tissue on which the therapeutic ultrasound energy is irradiated is increased to A° C., the medicine may effectively penetrate into the tissue upon which the therapeutic ultrasound is irradiated. In this manner, the therapeutic ultrasound energy may effectively deliver the medicine to a specific internal tissue of the object 30.

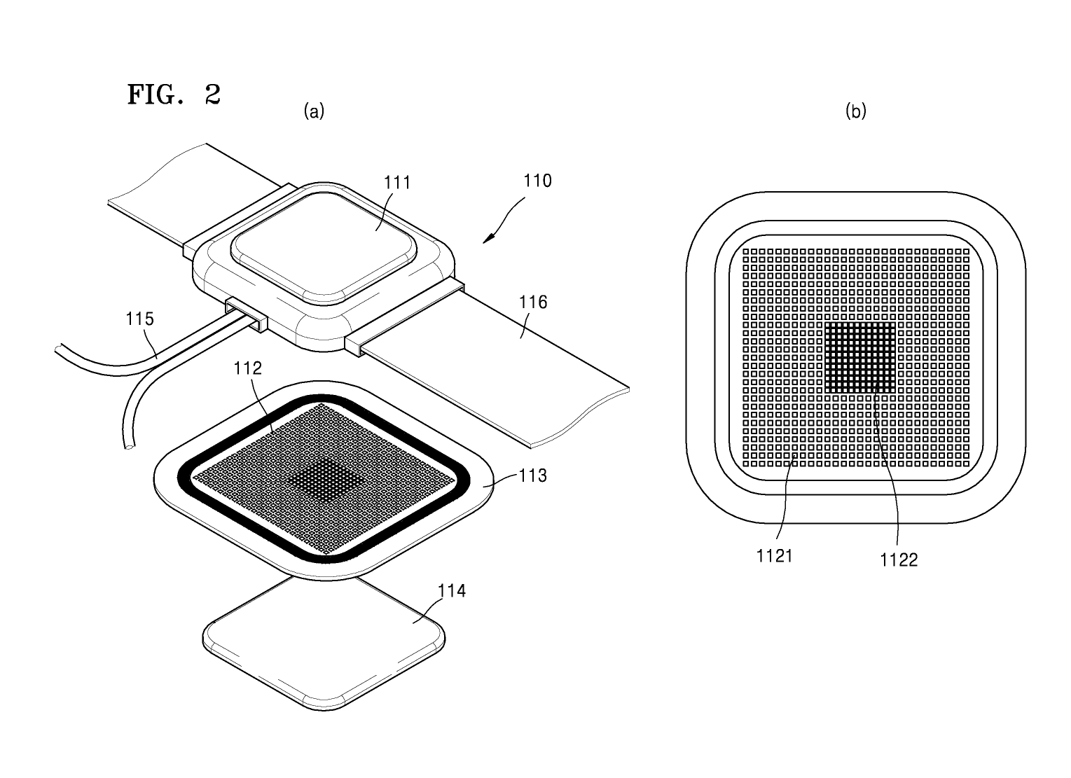

Operations of the ultrasound irradiation apparatus 110 irradiating the diagnostic ultrasound and the therapeutic ultrasound and receiving the echo signal corresponding to the diagnostic ultrasound will be described in detail with reference to FIG. 2.

FIG. 2 is a view illustrating an example of an ultrasound irradiation apparatus, according to an exemplary embodiment.

Drawing (a) of FIG. 2 illustrates the ultrasound irradiation apparatus 110, and drawing (b) of FIG. 2 illustrates a transducer set unit 112 included in the ultrasound irradiation apparatus 110.

Referring to drawing (a) of FIG. 2, the ultrasound irradiation apparatus 110 includes a case 111, the transducer set unit 112, an adhesive sticker 113, a contact pad 114, a data/power transmission cable 115, and a belt 116. The ultrasound irradiation apparatus 110 may be referred to as an applicator.

The case 111 protects the transducer set unit 112 against external shock.

The transducer set unit 112 includes a first set of first transducers and a second set of second transducers. Here, the first transducers irradiate the diagnostic ultrasound energy upon the object and receive the echo signal which corresponds to the irradiated diagnostic ultrasound energy. In addition, the second transducers irradiate the therapeutic ultrasound energy upon the region of interest. Hereinafter, referring to drawing (b) of FIG. 2, the transducer set unit 112 will be described in detail.

In drawing (b) of FIG. 2, the first transducers 1122 and the second transducers 1121 are illustrated.

The first transducers 1122 irradiate the diagnostic ultrasound energy upon the object. The first transducers 1122 receive the echo signal obtained as a result of the irradiated diagnostic ultrasound energy being reflected from internal tissues of the object.

Specifically, when the diagnostic ultrasound energy, which has a frequency which falls in the range of about 2 MHz to about 18 MHz, is irradiated by the first transducers 1122 upon a specific part in the object, the diagnostic ultrasound energy is partially reflected from layers among various tissues. In particular, the diagnostic ultrasound energy may be reflected from parts in the object where a change in density is generated (for example, blood cells in blood plasma and small tissues in organs). The reflected signals cause vibrations in the first transducers 1122, and the first transducers 1122 output electrical pulses in accordance with the vibrations.

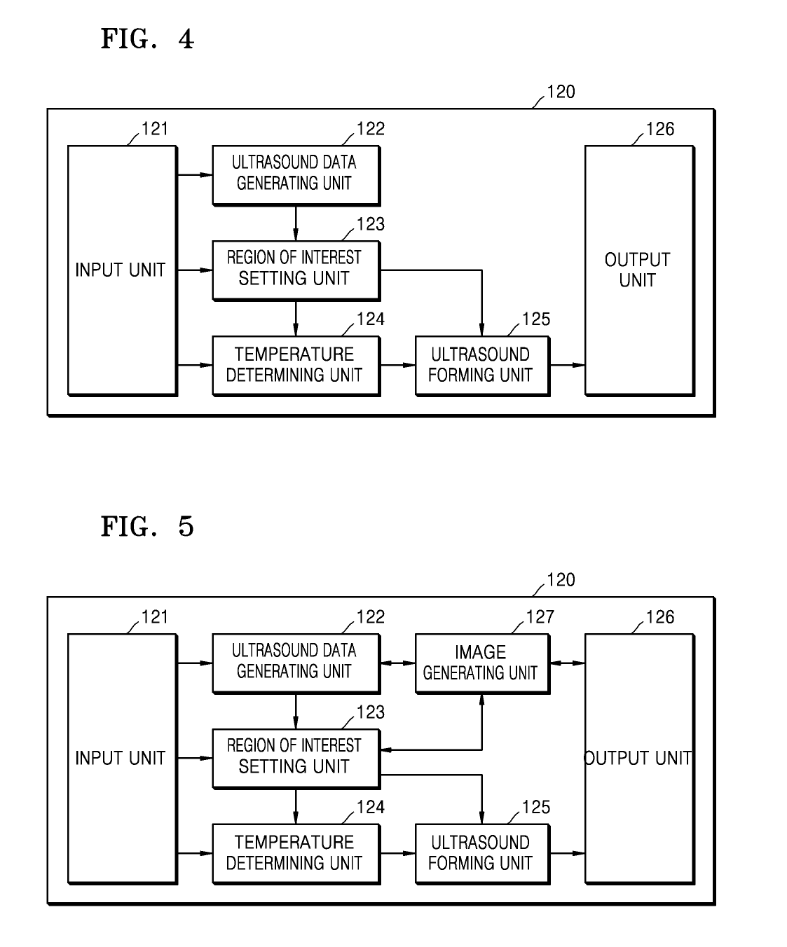

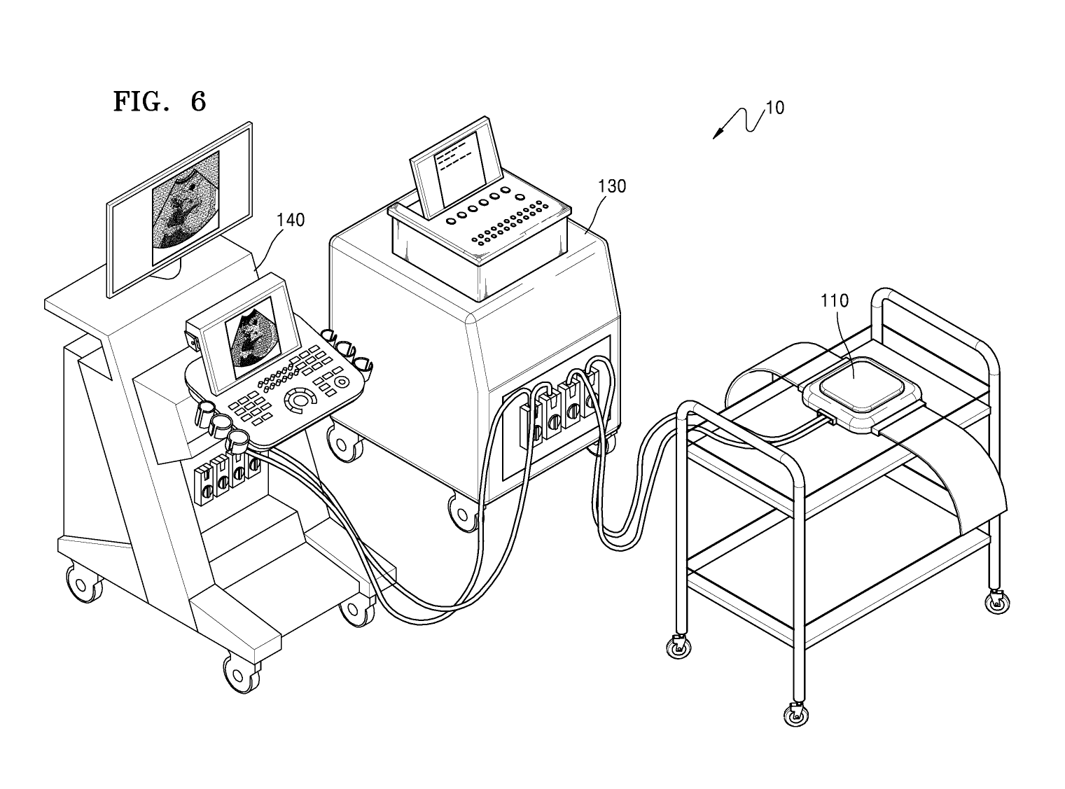

The second transducers 1121 irradiate the therapeutic ultrasound energy upon the region of interest which is determined based on a result of processing of the echo signal. Here, the region of interest is an internal region of the object determined by the ultrasound processing apparatus 120, and includes a position of a focus upon which the therapeutic ultrasound energy is to be irradiated. A method of the ultrasound processing apparatus 120 determining the region of interest and the second transducers 1121 generating the therapeutic ultrasound energy to be irradiated will be described in detail with reference to FIGS. 4 and 5.

The first transducers 1122 and the second transducers 1121, according to the exemplary embodiment, form respective two-dimensional array structures. For example, as illustrated in drawing (b) of FIG. 2, the first transducers 1122 and the second transducers 1121 may be arranged in the form of two-dimensional array. In drawing (b) of FIG. 2, the first transducers 1122 are positioned in the center of the arrangement of second transducers 1121. However, the present exemplary embodiment is not limited to the above. In particular, provided that the first transducers 1122 and the second transducers 1121 form the transducer set unit 112 of the two-dimensional array, there are no limitations on a positional relationship between the set of the first transducers 1122 and the set of the second transducers 1121.

As described above, the first transducers 1122 and the second transducers 1121 form a single transducer set unit 112 so that a user (for example, a doctor) may simultaneously diagnose and treat the object (for example, a patient). Specifically, after the second transducers 1121 commence irradiating the therapeutic ultrasound energy upon the region of interest, the user may check an operation result of the irradiated therapeutic ultrasound energy by using the diagnostic ultrasound energy irradiated by the first transducers 1122.

In addition, because the first transducers 1122 and the second transducers 1121 form the two-dimensional array structures, respectively, the first transducers 1122 and the second transducers 1121 may irradiate two-dimensional ultrasound signals and/or three-dimensional ultrasound signals.

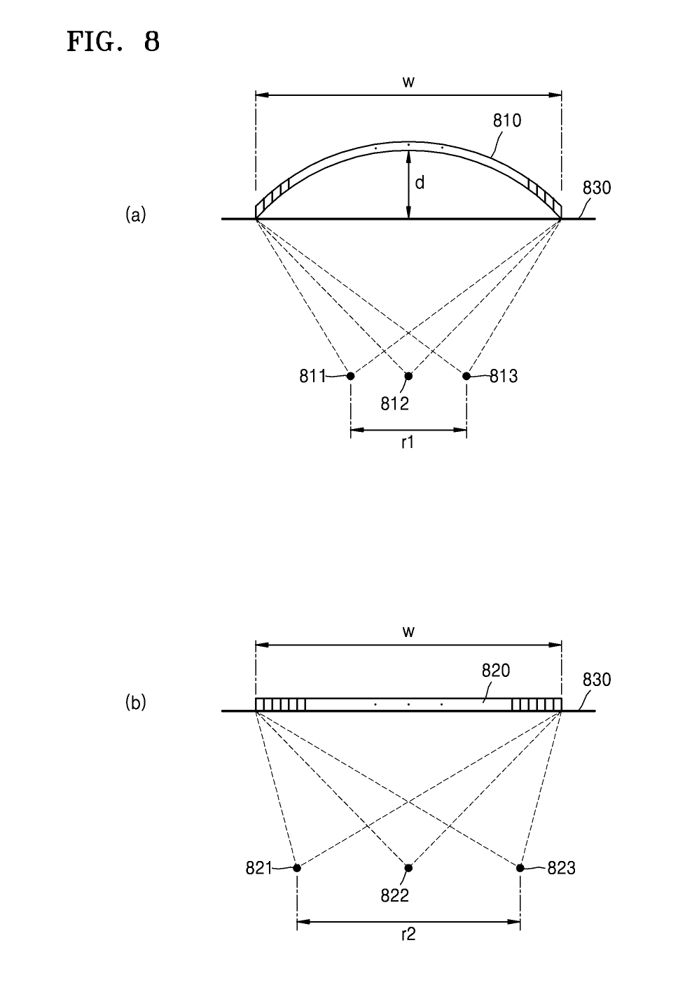

Conversely, the first transducers 1122 and the second transducers 1121, according to the exemplary embodiment, are arranged on a single plane. In this aspect, the transducer set unit 112 forms one single plane. As described above, since the first transducers 1122 and the second transducers 1121 form the two-dimensional array structures, respectively, the transducer set unit 112 may represent a rectangle on one plane. Hereinafter, the second transducers 1121 will be described in detail with reference to FIG. 8.

FIG. 8 is a view illustrating second transducers, according to an exemplary embodiment.

Drawing (a) of FIG. 8 illustrates an arrangement of common transducers 810 that irradiate the therapeutic ultrasound energy, and drawing (b) of FIG. 8 illustrates an arrangement of second transducers 820, according to an exemplary embodiment.

Referring to drawing (a) of FIG. 8, the transducers 810 that irradiate the therapeutic ultrasound energy are concavely arranged. Specifically, since the therapeutic ultrasound energy irradiated by the transducers 810 forms focuses 811, 812, and 813, the transducers 810 are commonly concavely arranged so as to easily form the focuses 811, 812, and 813. However, the concavely arranged transducers 810 have a narrow steering range. In particular, due to a concave geometric structure, a range r1 of the focuses 811, 812, and 813 that may be formed by the therapeutic ultrasound energy which is irradiated by the transducers 810 is limited.

In addition, when the transducers 810 are concavely arranged, all the transducers 810 may not be adhered to a surface 830 of the object. In particular, an empty space d is formed between the transducers 810 and the surface 830 of the object. At this time, due to the empty space d between the surface 830 of the object and the transducers 810, respective positions of the focuses 811, 812, and 813 formed by the therapeutic ultrasound irradiated by the transducers 810 may not coincide with those set by the ultrasound processing apparatus 120.

Referring to drawing (b) of FIG. 8, the second transducers 820 according to an exemplary embodiment are arranged on one single plane. Therefore, a range r2 (that is, a steering range) of focuses 821, 822, and 823 which is formed by the therapeutic ultrasound energy irradiated by the second transducers 820 is larger than the range r1 of the focuses 811, 812, and 813 formed by the therapeutic ultrasound energy irradiated by the transducers 810 illustrated in drawing (a) of FIG. 8. In particular, when it is assumed that the transducers 810 illustrated in drawing (a) of FIG. 8 and the second transducers 820 illustrated in drawing (b) of FIG. 8 have the same width w, the steering ranges have a relationship in which r1>r2. Therefore, although the region of interest has a large range, the therapeutic ultrasound energy irradiated by the second transducers 820 may form the focuses 821, 822, and 823 in the region of interest.

In addition, since the second transducers 820 are arranged on one single plane, all the second transducers 820 may be adhered to the surface 830 of the object. Therefore, an empty space is not formed between the second transducers 820 and the surface 830 of the object, or alternatively, to the extent that an empty space is formed, such an empty space is formed in a manner so as not to affect the formation of the focuses by therapeutic ultrasound energy. Therefore, positions of the focuses 821, 822, and 823 formed by the therapeutic ultrasound irradiated by the second transducers 820 coincide with positions set by the ultrasound processing apparatus 120.

As described above, since the second transducers 820 are arranged on a single plane, the second transducers 820 may form a large steering range and may correctly focus the therapeutic ultrasound energy on an intended position.

Conversely, the ultrasound irradiation apparatus 110 may be formed of a magnetic resonance (MR)-compatible material. In particular, the ultrasound irradiation apparatus 110 may be formed of a material that is not displayed in an MR image. Therefore, the ultrasound irradiation apparatus 110 and the ultrasound processing apparatus 120 may generate an image of the region of interest and simultaneously treat the region of interest together with a magnetic resonance imaging (MRI) apparatus.

Referring to drawing (a) of FIG. 2 again, the adhesive sticker 113 fixes the ultrasound irradiation apparatus 110 to the surface of the object. In particular, the adhesive sticker 113 fixes the ultrasound irradiation apparatus 110 to the surface of the object in order to cause the ultrasound irradiation apparatus 110 not to move on the surface of the object.

The contact pad 114 facilitates a contact between the object and each of the first transducers and the second transducers. In particular, the contact pad 114 facilitates an adhesion between the ultrasound transducer set unit 112, including the first transducers and the second transducers, and the surface of the object.

For example, the contact pad 114 may be implemented by a pocket including a specific material. Here, the material included in the contact pad 114 may include degassed water or deionized water, but is not limited thereto. In this aspect, any material that does not affect propagation of the diagnostic ultrasound energy and the therapeutic ultrasound energy irradiated by the transducer set unit 112 and propagation of the echo signal received by the transducer set unit 112 may correspond to the material included in the contact pad 114 without limitations.

In addition, the contact pad 114 may be implemented by a Mylar material or a thermal protection material. In particular, the pocket including the above-described specific material may be formed of the Mylar material or the thermal protection material.

The contact pad 114 not only facilitates an adhesion of the transducer set unit 112 to the surface of the object, but also cools the first transducers 1122 and the second transducers 1121 by circulation of the material included in the contact pad 114.

The data/power transmission cable 115 connects the ultrasound irradiation apparatus 110 to the ultrasound processing apparatus 120, and performs data transmission between the ultrasound irradiation apparatus 110 and the ultrasound processing apparatus 120. Here, data includes data corresponding to the echo signal obtained by the ultrasound irradiation apparatus 110 and data corresponding to the diagnostic ultrasound energy and the therapeutic ultrasound energy to be irradiated by the ultrasound irradiation apparatus 110.

In addition, the data/power transmission cable 115 supplies power to the ultrasound irradiation apparatus 110 in order to drive the first transducers 1122 and the second transducers 1121 included in the ultrasound irradiation apparatus 110.

The belt 116 fixes the ultrasound irradiation apparatus 110 to the object. In particular, the belt 116 fixes the ultrasound irradiation apparatus 110 to the object so as to cause the ultrasound irradiation apparatus 110 not to move on the surface of the object. More particularly, the contact pad 114 primarily fixes the ultrasound irradiation apparatus 110 with respect to the object, and the belt 116 secondarily fixes the ultrasound irradiation apparatus 110 with respect to the object.

As described above, since the contact pad 114 and the belt 116 fix the ultrasound irradiation apparatus 110 to the object, the ultrasound irradiation apparatus 110 may correctly irradiate the therapeutic ultrasound on the region of interest even when the object moves.

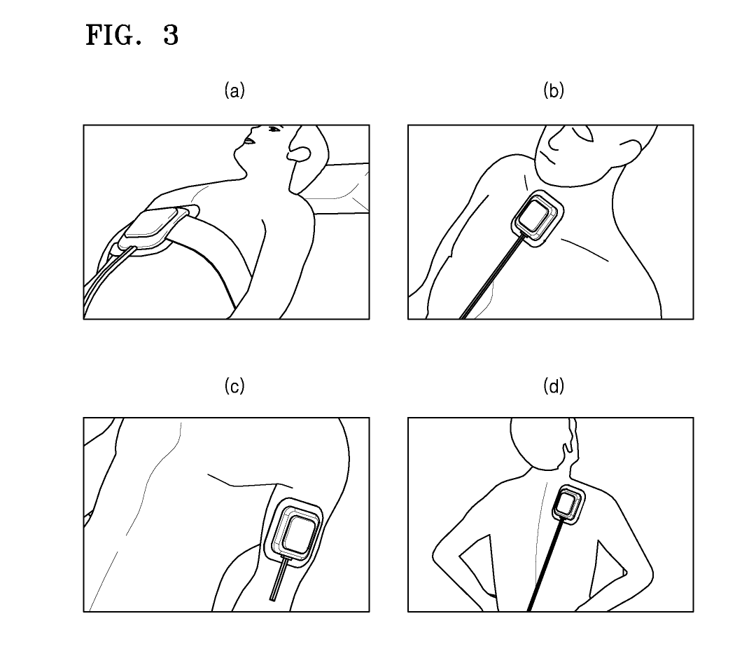

FIG. 3 is a view illustrating examples of implementing an ultrasound irradiation apparatus, according to an exemplary embodiment.

Referring to drawings (a), (b), (c), and (d) of FIG. 3, examples in which the ultrasound irradiation apparatus is adhered to the object are illustrated. When it is assumed that the object is a patient, the ultrasound irradiation apparatus may be adhered to any suitable part of the object, including a chest (drawing (a) of FIG. 3), a neck (drawing (b) of FIG. 3), an arm (drawing (c) of FIG. 3), and a back (drawing (d) of FIG. 3). In addition, referring to drawings (b), (c), and (d) of FIG. 3, when the parts to which the ultrasound irradiation apparatus is adhered are parts with small amounts of movement, the ultrasound irradiation apparatus may be adhered to the object by using only the adhesive sticker without the belt.

The ultrasound irradiation apparatus may have various sizes. For example, referring to drawing (a) of FIG. 3, when the diagnostic ultrasound energy and the therapeutic ultrasound energy are to be irradiated on a wide region of the chest of the object, a large ultrasound irradiation apparatus may be adhered to the chest of the object. In addition, referring to drawing (b) of FIG. 3, when the diagnostic ultrasound energy and the therapeutic ultrasound energy are to be irradiated on a narrow region of the neck of the object, a small ultrasound irradiation apparatus may be adhered to the neck of the object.

Here, the ultrasound irradiation apparatus may have various sizes in accordance with the number of first transducers and the number of second transducers included in the ultrasound irradiation apparatus.

Referring to FIG. 1 again, the ultrasound processing apparatus 120 determines the region of interest on which the therapeutic ultrasound energy is to be irradiated by using the echo signal received from the ultrasound irradiation apparatus 110. The ultrasound processing apparatus 120 determines a temperature of the region of interest to be increased by the irradiation of the therapeutic ultrasound energy. The ultrasound processing apparatus 120 generates the therapeutic ultrasound energy by using information which relates to a position of the region of interest and information which relates to the determined temperature. The ultrasound processing apparatus 120 transmits a signal which corresponds to the therapeutic ultrasound energy to the ultrasound irradiation apparatus 110. The operations performed by the ultrasound processing apparatus 120 will be described in detail with reference to FIGS. 4 and 5.

FIG. 4 is a block diagram illustrating an example of an ultrasound processing apparatus 120, according to an exemplary embodiment.

Referring to FIG. 4, the ultrasound processing apparatus 120 includes an input unit (also referred to herein as an “input device”) 121, an ultrasound data generating unit (also referred to herein as an “ultrasound data generator”) 122, a region of interest setting unit (also referred to herein as a “region of interest setter” and/or as a “region of interest determiner”) 123, a temperature determining unit (also referred to herein as a “temperature determiner”) 124, an ultrasound forming unit (also referred to herein as an “ultrasound former” and/or as an “ultrasound energy generator”) 125, and an output unit (also referred to herein as an “output device”) 126.

In the ultrasound processing apparatus 120 illustrated in FIG. 4, only elements related to the present exemplary embodiment are illustrated. Therefore, it will be understood by those of ordinary skill in the art that other general-purpose elements than the elements illustrated in FIG. 4 may be further included.

In addition, the input unit 121, the ultrasound data generating unit 122, the region of interest setting unit 123, the temperature determining unit 124, the ultrasound forming unit 125, and the output unit 126 of the ultrasound processing apparatus 120 illustrated in FIG. 4 may correspond to one or a plurality of processors. The processor may be implemented by array of a plurality of logic gates and may be implemented by a combination of a general-purpose microprocessor and a memory in which microprocessor executable programs are recorded. In addition, it will be understood by those of ordinary skill in the art that the processor may be implemented by another type of hardware.

The input unit 121 receives data corresponding to the echo signal from the ultrasound irradiation apparatus 110. In addition, the input unit 121 receives input information from the user. Here, the input information refers to information which relates to the region of interest set by the user or information which relates to the temperature determined by the user.

For example, the input unit 121 performs functions of a communication interface unit and a user interface unit. Here, the communication interface unit may include any one or more of a modem used for data transmission and reception, a network module for access to a network, and a universal serial bus (USB) host module for forming a data movement channel with respect to a mobile storage medium. In addition, the user interface unit may include input apparatuses such as any one or more of a mouse, a keyboard, and a touch screen and a software module for driving the input apparatuses.

The ultrasound data generating unit 122 generates ultrasound data by using the echo signal transmitted from the input unit 121.

In particular, the ultrasound data generating unit 122 converts the received analog echo signal from analog to digital in order to generate a digital signal. The ultrasound data generating unit 122 performs reception beamforming on the digital signal to form a reception focusing signal, and generates ultrasound data by using the reception focusing signal. Here, the ultrasound data may include a radio frequency (RF) signal, but is not limited thereto.

The region of interest setting unit 123 sets the region of interest by using the echo signal. Here, the region of interest refers to a target region on which the second transducers included in the ultrasound irradiation apparatus 110 focus the therapeutic ultrasound energy, which target region may include a lesion.

As an example, the region of interest setting unit 123 may set the region of interest by using the ultrasound data transmitted from the ultrasound data generating unit 122. The ultrasound data includes information which relates to the internal tissues of the object. In particular, the ultrasound data includes information which relates to whether a lesion such as a cancer is included in the internal tissues of the object, as well as information which relates to sizes and shapes of the internal tissues of the object.

Therefore, the region of interest setting unit 123 may set the region of interest upon which the therapeutic ultrasound energy is to be irradiated by using the ultrasound data. Here, the therapeutic ultrasound focused on the region of interest increases the temperature of the region of interest so that the medicine administered to the object may effectively penetrate into the region of interest.

As another example, the region of interest setting unit 123 may set the region of interest to correspond to the input information provided by the user. In particular, the region of interest setting unit 123 may set the region of interest by using the input information which is provided by the user and received via the input unit 121. Here, the input information of the user refers to information that represents the region of interest set by the user.

The temperature determining unit 124 determines the threshold temperature of the region of interest to be reached as a result of the therapeutic ultrasound energy being irradiated. Here, the threshold temperature to be determined may refer to a predetermined temperature at which the medicine previously administered to the object may effectively penetrate into the region of interest, but is not limited thereto.

As an example, the temperature determining unit 124 may automatically determine the threshold temperature without user intervention so as to correspond to a kind of the medicine previously administered to the object. For example, the temperature determining unit 124 may determine the temperature by using information which relates to a correlation between medicine previously stored in a storage unit (not shown) and an activation temperature.

As another example, the temperature determining unit 124 may determine the threshold temperature so to correspond to the input information provided by the user. In particular, the temperature determining unit 124 may determine the threshold temperature by using the input information provided by the user and received via the input unit 121. Here, the input information of the user refers to information that represents a target temperature set by the user.

The ultrasound forming unit 125 generates the therapeutic ultrasound energy to be irradiated on the region of interest by using information which relates to the position of the region of interest and information which relates to the determined threshold temperature. In particular, the ultrasound forming unit 125 generates the therapeutic ultrasound energy by using the information which relates to the position of the region of interest received from the region of interest setting unit 123 and the information which relates to the threshold temperature received from the temperature determining unit 124.

For example, the ultrasound forming unit 125 calculates an irradiation intensity and an irradiation time of the therapeutic ultrasound energy required for increasing the temperature of the region of interest to the threshold temperature determined by the temperature determining unit 124. Then, the ultrasound forming unit 125 may perform transmission beamforming by using the calculated intensity and time to generate a signal corresponding to the therapeutic ultrasound energy. Here, the signal corresponding to the therapeutic ultrasound energy includes information which relates to transducers from which the therapeutic ultrasound energy is to be irradiated among the second transducers and information which relates to a time for which the therapeutic ultrasound energy is to be irradiated.

In addition, the ultrasound forming unit 125 generates a signal corresponding to the diagnostic ultrasound energy to be irradiated by the first transducers included in the ultrasound irradiation apparatus 110.

The output unit 126 transmits the signal corresponding to the therapeutic ultrasound energy to the ultrasound irradiation apparatus 110. In particular, the output unit 126 transmits the signal corresponding to the therapeutic ultrasound energy which is received from the ultrasound forming unit 125 to the ultrasound irradiation apparatus 110. In addition, the output unit 126 transmits the signal corresponding to the diagnostic ultrasound energy which is received from the ultrasound forming unit 125 to the ultrasound irradiation apparatus 110.

For example, the output unit 126 may perform a function of a communication interface, and the communication interface unit is as described above with reference to the input unit 121.

FIG. 5 is a block diagram illustrating another example of an ultrasound processing apparatus 120, according to an exemplary embodiment.

Referring to FIG. 5, the ultrasound processing apparatus 120 includes an image generating unit (also referred to herein as an “image generator”) 127 as well as the input unit 121, the ultrasound data generating unit 122, the region of interest setting unit 123, the temperature determining unit 124, the ultrasound forming unit 125, and the output unit 126.

In the ultrasound processing apparatus 120 illustrated in FIG. 5, only elements related to the present exemplary embodiment are illustrated. Therefore, it will be understood by those of ordinary skill in the art that other general-purpose elements than the elements illustrated in FIG. 5 may be further included.

In addition, the input unit 121, the ultrasound data generating unit 122, the region of interest setting unit 123, the temperature determining unit 124, the ultrasound forming unit 125, the output unit 126, and the image generating unit 127 of the ultrasound processing apparatus 120 illustrated in FIG. 5 may correspond to one or a plurality of processors. The processor may be implemented by array of a plurality of logic gates and may be implemented by a combination of a general-purpose microprocessor and a memory in which microprocessor executable programs are recorded. In addition, it will be understood by those of ordinary skill in the art that the processor may be implemented by another type of hardware.

Conversely, detailed contents of the input unit 121, the ultrasound data generating unit 122, the region of interest setting unit 123, the temperature determining unit 124, the ultrasound forming unit 125, and the output unit 126 of the ultrasound processing apparatus 120 illustrated in FIG. 5 are as described above with reference to FIG. 4. Therefore, detailed description thereof will be omitted.

The image generating unit 127 generates an image that represents the object by using information included in the echo signal. In particular, the image generating unit 127 generates the image that represents the object by using the ultrasound data received from the ultrasound data generating unit 122. Here, the image that represents the object includes one or more images that represent the shapes and sizes of the internal tissues of the object. For example, the image that represents the object may include a Doppler mode image, a B mode image, and an elasticity image and may be a two-dimensional image or three-dimensional image.

In addition, the image generating unit 127 may generate a real time image that represents the object by using the information included in the echo signal corresponding to the diagnostic ultrasound energy irradiated upon the object in real time.

In addition, the image generating unit 127 generates an image that represents a change in temperature of the region of interest by using the echo signal corresponding to the diagnostic ultrasound energy irradiated upon the region of interest after a commencement of irradiating the therapeutic ultrasound energy upon the region of interest. In particular, the image generating unit 127 may generate the image that represents the change in temperature of the region of interest by using the ultrasound data received from the ultrasound data generating unit 122. An example in which the image generating unit 127 generates the image that represents the change in temperature of the region of interest is as follows.

First, the image generating unit 127 obtains first temperature information which relates to the region of interest by using the echo signal corresponding to the diagnostic ultrasound energy irradiated upon the region of interest before a commencement of irradiating the therapeutic ultrasound energy upon the region of interest. Here, the first temperature information is obtained based on an amplitude of the echo signal and/or a phase of the echo signal.

Then, the image generating unit 127 obtains second temperature information which relates to the region of interest by using the echo signal corresponding to the diagnostic ultrasound energy irradiated upon the region of interest after the commencement of irradiating the therapeutic ultrasound energy upon the region of interest. Here, the second temperature information is obtained based on the amplitude of the echo signal and/or a phase of the echo signal.

Then, the image generating unit 127 compares the first temperature information with the second temperature information. An example in which the image generating unit 127 compares the first temperature information with the second temperature information is as follows.

As an example, the image generating unit 127 compares an RF signal included in the first temperature information with an RF signal included in the second temperature information in order to detect a part of the RF signal included in the second temperature information in which an amplitude is changed. Then, the image generating unit 127 obtains information (for example, a change in temperature value) that represents the change in temperature of the region of interest corresponding to the detected degree of change in amplitude by using a mapping table stored in the storage unit (not shown).

Here, the mapping table may include respective changes in amplitude values of a plurality of predetermined echo signals and corresponding changes in temperature values mapped to the changes in amplitude values. In the mapping table, the change in temperature value mapped to one change in amplitude value refers to a change in temperature value of the region of interest estimated from the change in amplitude value of the region of interest.

As another example, the image generating unit 127 compares the RF signal included in the first temperature information with the RF signal included in the second temperature information in order to detect a part of the RF signal included in the second temperature information in which a phase is changed. Then, the image generating unit 127 obtains information (for example, a change in temperature value) that represents the change in temperature of the region of interest corresponding to the detected degree of change in phase by using the mapping table stored in the storage unit (not shown).

Here, the mapping table may include respective changes in phase values of a plurality of predetermined echo signals and corresponding changes in temperature values mapped to the changes in phase values. In the mapping table, the change in temperature value mapped to one change in phase value refers to the change in temperature value of the region of interest estimated from the change in phase value of the region of interest.

The image generating unit 127 generates a temperature image of the region of interest based on a result of the comparison between the first temperature information and the second temperature information. Here, the temperature image refers to an image that represents degrees of change in temperature of the region of interest by using different brightness values or colors.

Conversely, the ultrasound forming unit 125 may generate the therapeutic ultrasound energy to be irradiated upon the region of interest by using the information which relates to the change in temperature of the region of interest. For example, the ultrasound forming unit 125 receives the information which relates to the change in temperature of the region of interest from the image generating unit 127. When a current temperature of the region of interest is lower than a predetermined temperature, the ultrasound forming unit 125 may regenerate therapeutic ultrasound energy so as to have a higher intensity and/or a longer irradiation time than the previously generated therapeutic ultrasound energy. In addition, when the current temperature of the region of interest is equal to or higher than the predetermined threshold temperature, the ultrasound forming unit 125 may stop generating the therapeutic ultrasound energy.

FIG. 6 is a block diagram illustrating another example of an ultrasound system, according to an exemplary embodiment.

Referring to FIG. 6, the ultrasound system 10 includes the ultrasound irradiation apparatus 110, a first ultrasound processing apparatus 140, and a second ultrasound processing apparatus 130.

The ultrasound irradiation apparatus 110 according to the exemplary embodiment may be connected to the previously provided first ultrasound processing apparatus 140. For example, when it is assumed that the previously provided first ultrasound processing apparatus 140 is configured for generating an ultrasound image by using the echo signal, the second ultrasound processing apparatus 130 which is configured for generating the therapeutic ultrasound energy is connected to the first ultrasound processing apparatus 140, and the ultrasound irradiation apparatus 110 is connected to the already-connected first and second ultrasound processing apparatuses 130 and 140 so that one ultrasound system 10 may be formed.

In this aspect, similarly as the ultrasound processing apparatus 120 described in detail with reference to FIGS. 1, 4, and 5, one ultrasound processing apparatus 120 may perform all the above-described functions. However, the first ultrasound processing apparatus 140 and the second ultrasound processing apparatus 130 may be connected to perform the same function as that of the ultrasound processing apparatus 120.

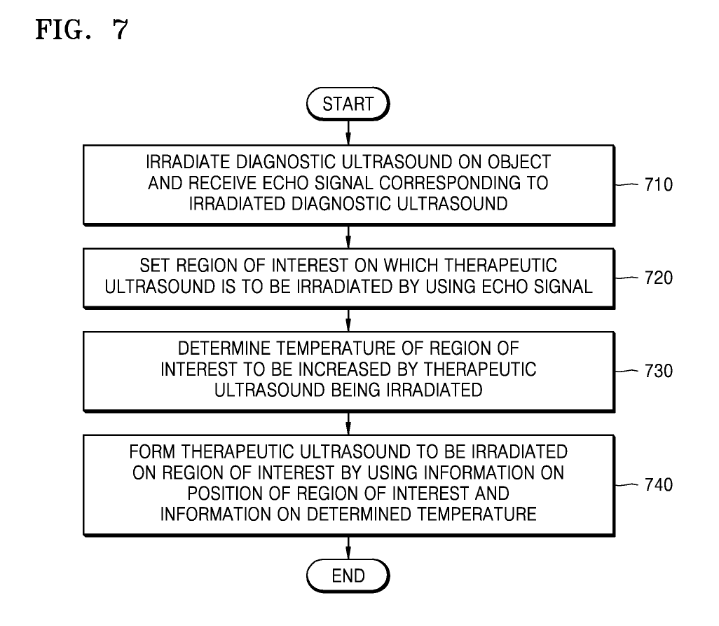

FIG. 7 is a flowchart illustrating an example of a method for irradiating ultrasound energy upon a region of interest, according to an exemplary embodiment.

Referring to FIG. 7, the method for irradiating the ultrasound energy upon the region of interest includes operations which are serially processed by the ultrasound system 10 or the ultrasound processing apparatus 120 illustrated in FIGS. 1, 4, and 5. Therefore, the above-described contents of the ultrasound system 10 or the ultrasound processing apparatus 120 illustrated in FIGS. 1, 4, and 5, although omitted hereinafter, may be applied to the method for irradiating the ultrasound on the region of interest of FIG. 7.

In operation 710, the ultrasound irradiation apparatus 110 irradiates the diagnostic ultrasound energy upon the object and receives the echo signal corresponding to the irradiated diagnostic ultrasound energy. Here, the diagnostic ultrasound energy may refer to ultrasound energy to be used for generating an image that represents the shapes of the internal tissues of the object 30, and may refer to ultrasound energy to be used for generating an image that represents the change in temperature of the region of interest.

In operation 720, the ultrasound processing apparatus 120 determines the region of interest upon which the therapeutic ultrasound energy is to be irradiated by using the echo signal. In particular, the ultrasound data generating unit 122 generates the ultrasound data by using the echo signal received from the input unit 121. The region of interest setting unit 123 determines the region of interest by using he echo signal. Here, the region of interest refers to the target region upon which the second transducers included in the ultrasound irradiation apparatus 110 focus the therapeutic ultrasound energy, which region of interest may include the lesion.

In operation 730, the ultrasound processing apparatus 120 determines the threshold temperature of the region of interest to be reached as a result of the therapeutic ultrasound energy being irradiated. In particular, the temperature determining unit 124 determines the threshold temperature of the region of interest to be reached by using the therapeutic ultrasound energy being irradiated. Here, the threshold temperature to be determined may refer to a predetermined temperature at which the medicine previously administered to the object may effectively penetrate into the region of interest, but is not limited thereto.

In operation 740, the ultrasound processing apparatus 120 generates the therapeutic ultrasound energy to be irradiated upon the region of interest by using the information which relates to the position of the region of interest and the information which relates to the determined threshold temperature. In particular, the ultrasound forming unit 125 generates the therapeutic ultrasound energy by using the information which relates to the position of the region of interest received from the region of interest setting unit 123 and the information which relates to the threshold temperature received from the temperature determining unit 124.

Conversely, the above-described method may be created by computer executable programs and may be implemented by a general-purpose digital computer that operates the programs by using a transitory or non-transitory computer readable recording medium. In addition, a structure of data used by the above-described method may be recorded in the computer readable recording medium via any one or more of various units. The computer readable recording medium includes a storage medium such as, for example, any one or more of a magnetic storage medium (for example, a read only memory (ROM), a random access memory (RAM), an USB, a floppy disk, and a hard disk), and/or an optical reading medium (for example, a CD-ROM and a digital versatile disk (DVD)).

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the present inventive concept as defined by the following claims.

What is claimed is:

1. An ultrasound irradiation apparatus, comprising:
first transducers configured to irradiate first ultrasound energy upon an object and to receive an echo signal which corresponds to the irradiated first ultrasound energy;
second transducers configured to irradiate second ultrasound energy upon a region of interest which is determined based on a result of processing of the received echo signal; and
a contact pad configured to facilitate a contact between the first transducers and the object, and to facilitate a contact between the second transducers and the object.

2. The ultrasound irradiation apparatus of claim 1, wherein the first transducers form a first two-dimensional array structure, and wherein the second transducers form a second two-dimensional array structure.

3. The ultrasound irradiation apparatus of claim 1, wherein the first transducers are arranged in a first plane, and wherein the second transducers are arranged in a second plane.

4. The ultrasound irradiation apparatus of claim 1, further comprising a belt configured to fix the ultrasound irradiation apparatus to the object.

5. The ultrasound irradiation apparatus of claim 1, wherein the second ultrasound energy comprises an amount of ultrasound energy which is sufficient for increasing a temperature of the region of interest to a predetermined temperature in order to facilitate a penetration of medicine into the region of interest.

6. The ultrasound irradiation apparatus of claim 1, wherein the first ultrasound energy comprises ultrasound energy to be used for generating an image that represents at least one from among the object and a change in temperature of the region of interest.

7. The ultrasound irradiation apparatus of claim 1,
wherein the contact pad comprises at least one from among degassed water and deionized water, and
wherein a material of the contact pad comprises at least one from among a Mylar material and a thermal protection material.

8. A method for irradiating ultrasound energy upon a region of interest, the method comprising:
irradiating first ultrasound energy upon an object and receiving an echo signal which corresponds to the irradiated first ultrasound energy;
determining a region of interest upon which second ultrasound energy is to be irradiated by using the received echo signal;
determining a threshold temperature of the region of interest to be reached as a result of the second ultrasound energy being irradiated; and
generating a sufficient amount of the second ultrasound energy to be irradiated upon the region of interest by using information relating to a position of the determined region of interest and information relating to the determined threshold temperature.

9. The method of claim 8, further comprising generating an image that represents a change in temperature of the region of interest by using the received echo signal after commencing irradiating the second ultrasound energy upon the region of interest.

10. The method of claim 9, wherein the generating the sufficient amount of the second ultrasound energy comprises using information which relates to the change in temperature of the region of interest.

11. The method of claim 9, wherein the generating the image that represents the change in temperature of the region of interest comprises:
obtaining first temperature information which relates to the region of interest by using the received echo signal prior to the commencing irradiating the second ultrasound energy upon the region of interest;
obtaining second temperature information which relates to the region of interest by using the received echo signal after the commencing irradiating the second ultrasound energy upon the region of interest; and
generating the image that represents the change in temperature of the region of interest based on a result of a comparison between the obtained first temperature information and the obtained second temperature information.

12. The method of claim 11, wherein each of the first temperature information and the second temperature information is obtained based on at least one from among an amplitude of the received echo signal and a phase of the received echo signal.

13. The method of claim 8, further comprising generating an image that represents the object by using information included in the received echo signal.

14. The method of claim 8, wherein the second ultrasound energy comprises an amount of ultrasound energy which is sufficient for increasing a temperature of the region of interest to a predetermined temperature in order to facilitate a penetration of medicine into the region of interest.

15. A non-transitory computer readable storage medium storing a computer program for executing the method of claim 8 in a computer.

16. An ultrasound system, comprising:
an ultrasound irradiation apparatus which includes first transducers configured to irradiate first ultrasound energy upon an object and to receive an echo signal which corresponds to the irradiated first ultrasound energy and second transducers configured to irradiate second ultrasound energy upon a region of interest; and
an ultrasound processing apparatus configured to determine the region of interest upon which the second ultrasound energy is to be irradiated by using the received echo signal, to determine a threshold temperature of the region of interest to be reached as a result of the second ultrasound energy being irradiated, to generate the second ultrasound energy by using information which relates to a position of the determined region of interest and information which relates to the determined threshold temperature, and to transmit a signal which corresponds to the generated second ultrasound energy to the ultrasound irradiation apparatus.
